(19) United States
(12) Patent Application Publication
(10) Pub. No.: US 2011/0313337 A1
(43) Pub. Date: Dec. 22, 2011

(54) BACK BRACE

(75) Inventors: Derek John Goldfinch, Auckland (NZ); Mark Appleton Hildesley, Auckland (NZ); Donald Joseph Woodward, Auckland (NZ)

(73) Assignee: SUMSMART IP LIMITED, Albany, Auckland (NZ)

(21) Appl. No.: 13/148,480

(22) PCT Filed: Feb. 5, 2010
(86) PCT No.: PCT/IB2010/050525

§ 371 Date: Aug. 9, 2011

(30) Foreign Application Priority Data
Feb. 9, 2009 (NZ) 574785

(51) Int. Cl. A61F 5/01

(52) U.S. Cl. 602/19

(57) ABSTRACT

A back brace including two support arms, each support arm including an underarm support located at one distal end, said underarm support is configured, when in use, to support an underarm of a user; wherein each support arm includes a hinge section, located close to or at the opposite distal end to that support arm's underarm support, each said hinge section incorporates a bias means configured to bias said support arm towards alignment

### FIELD OF THE INVENTION

The present invention is a back brace or support that reduces the vertical load component on the spine.

### BACKGROUND ART

Back problems, and more particularly lower back problems, with the associated pain and discomfort are often related to excessive wear or injury and can be difficult to manage. The constant and/or extreme pain often associated with back problems can prevent those suffers from leading a normal life or even functioning at all. This pain can also cause those suffering to adopt an abnormal posture to alleviate it. This unnatural posture can cause uneven or unnatural wear on vertebrae, uneven loading on the back muscles and/or lead to neck/hip/knee or shoulder problems.

One solution is to alleviate the pain by taking pain killers. This does nothing to prevent further damage to the back which can in some cases lead to reduced mobility and/or more serious problems arising. In addition the body's tolerance for many pain killers increases over time which necessitates ever increasing doses for the same effect, it can also lead to addiction and other complicating factors. For this reason they are not generally a long term solution, and can in fact make the problem far worse by obscuring the pain as more damage is done.

A variety of back braces and supports exist, some of these incorporate pads held in place by belts around the hips. These devices are useful but immobilise the lower back to alleviate the pain and prevent further damage. This immobilisation can prevent normal movement and turning.

An alternative to the pads held in place by belts and straps is provided by devices that include a crutch under each arm, each crutch is attached to a belt or strap around the hip of the user. Early versions of these devices supported under one arm rather than both and/or incorporated rigidly fixed crutches which prevented any twisting or turning motions by the user. Others are bulky and cumbersome, which reduces their usefulness, certainly it prevents them being discreet.

The underarm support of crutches can often become uncomfortable to wear for long periods; this can limit the usefulness of underarm support devices. In addition the use of straps over the shoulders to keep the crutch under the arm can prevent users from stretching upwards.

### OBJECT OF THE INVENTION

It would be advantageous if a back support device could be found that overcame one or more of the disadvantages highlighted above, or provided the consumer with a useful choice.

### DISCLOSURE OF THE INVENTION

The present invention provides a back brace including two support arms, each support arm including an underarm support located at one distal end, said underarm support is configured, when in use, to support an underarm of a user; wherein each support arm includes a hinge section, located close to or at the opposite distal end to that support arm's underarm support, each said hinge section incorporates a bias means configured to bias said support arm towards alignment.

Preferably the bias means is a spring loaded or elastic bias means.

Preferably the distal end of the support arm closest to the hinge includes either a belt connection plate or engagement section configured to releasably engage with a user's hip.

Preferably the back brace includes a belt releasably attached to each support arm. It is preferred that the belt includes a connection means configured to releasably connect the free ends of the belt. Preferably the connection means is selected from the list consisting of a buckle, a magnetic catch, hook and loop connectors fixed to each free end and a spring loaded connector.

Preferably each support arm includes a connection section that is releasably attached to its respective underarm support. In a highly preferred form the connection section includes an exposed end that is configured and dimensioned to be inserted into a matching engagement socket. Preferably this engagement socket is a hole of fixed depth.

Preferably each underarm support is trilobed, essentially equilateral in form, with a smooth symmetrical primary curved section, joining each adjacent lobe.

Alternatively, where the underarm support is not trilobed the underarm support includes a smooth primary curved section configured to engage with the underarm of the user. Preferably this smooth primary curved section is symmetrical.

In a highly preferred form each primary curved section includes an essentially centrally located secondary curved section which forms an indentation in the surface of the primary curved section. It is preferred that the radius of the primary curved section is about 1.1 to 2 times the radius of the secondary curved section. In a highly preferred form the depth of the indentation formed by the secondary curved section in the primary curved section is between 5% and 15% of its radius. In a highly preferred form the depth of the indentation is 10%.

In a further highly preferred form, where the underarm support is trilobed, each underarm support is dished, the lobes are co-planar with the centre of the underarm support being distanced from that plane.

Preferably, if the underarm support is trilobed, then each lobe includes an engagement socket configured to releasably engage with the exposed end of the matching connection section. In a highly preferred form each engagement socket is a hole extending along the centreline of the associated lobe. Preferably each of the engagement sockets is a different depth. Preferably each connection section includes an adjustment section configured to allow a user to adjust the length of associated support arm. In a highly preferred form the exposed end includes an expanded section of resilient material configured to be inserted into the associated engagement socket and form a releasably resilient connection with the underarm support.

Preferably the hinge includes a stop means that prevents the associated support arm from bending in one direction. Preferably this direction is backwards. In one preferred form the bias means is configured to allow the associated support arm to move in two opposite directions and return it to a central position.

Preferably each support arm includes a swivel section configured to allow axial rotation of the underarm support relative to the associated support arm. In a highly preferred form this swivel section is formed by the exposed end and the associated engagement socket.

### DESCRIPTION OF THE DRAWINGS

By way of example only a preferred embodiment of the back brace will now be described in detail with reference to the accompanying drawings in which:

FIG. 1 is a front view of the back brace in the rest position;

FIG. 2 is a side view of the back brace in the rest position;

FIG. 3 is a side view of a support arm in the rest position;

FIG. 4 is a cross sectional view through A-A of the first underarm support;

FIG. 5 is a side view of a further embodiment of a support arm with the belt connection plate replaced with a moulded section configured to engage with the top of a users hip.

FIG. 6 is a side view of a further embodiment of a support arm where the underarm support is not trilobed.

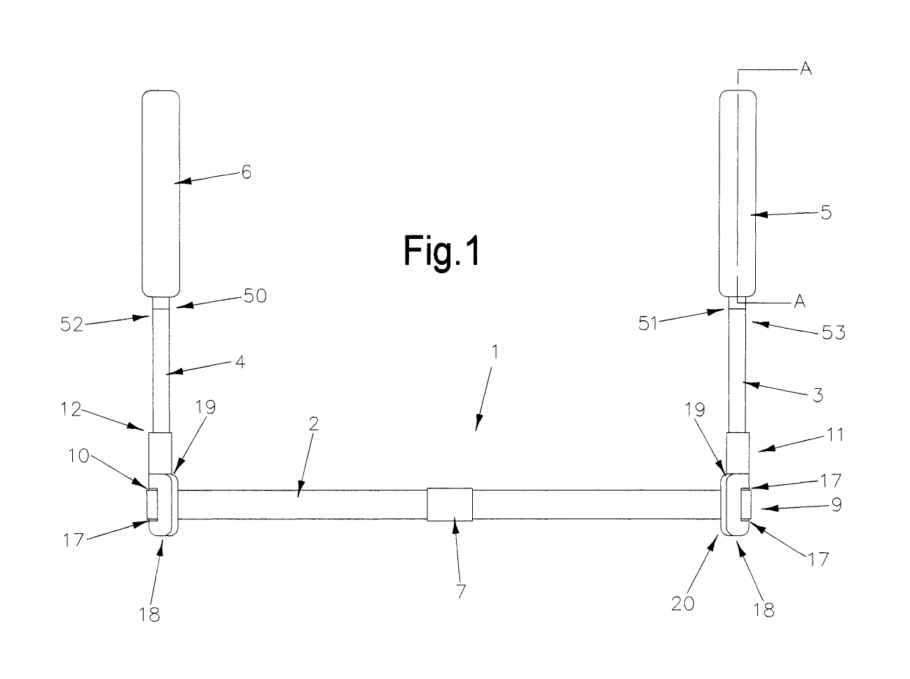

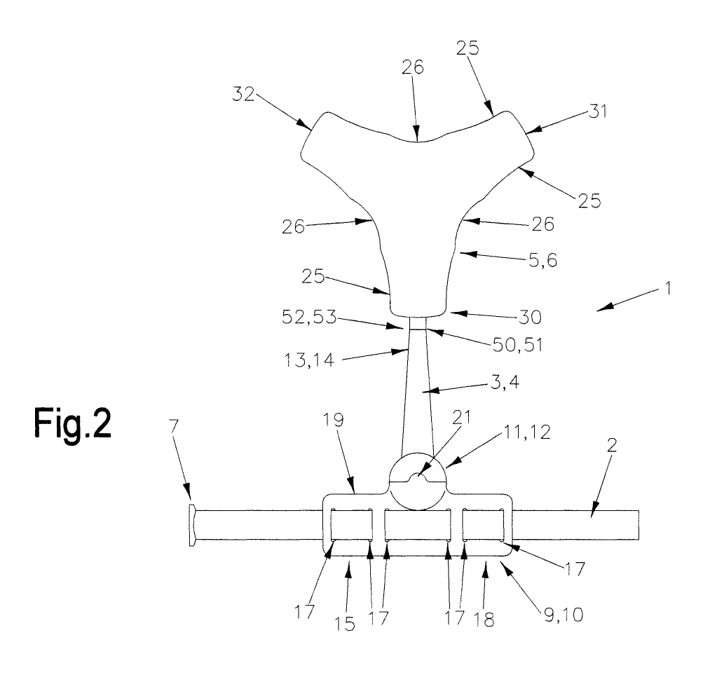

Referring to FIGS. 1 and 2, a back brace (1) including a belt (2) and two support arms (3,4), each with a releasably attached underarm support (5,6), is shown. The belt (2) includes a buckle (7) or similar known device that allows the free ends of the belt (2) to be releasably connected.

Each support arm (3,4) is releasably attached to, and extends away from, the belt (2). Each support arm (3,4) extends from the belt (2) in the same direction. The first support arm (3) is located on the opposite side of the back brace (1) to the second support arm (4).

Each support arm (3,4) further includes a belt connection plate (9,10), a hinge section (11,12) and a connection section (13,14).

Each support arm (3,4) is essentially a mirror image of the other, thus we shall describe the support arms (3,4) by describing the first support arm (3) in detail.

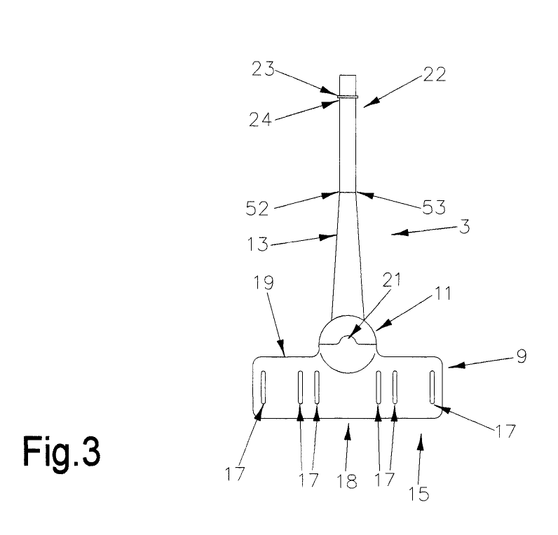

Referring to FIG. 3, the first support arm (3) is shown without the first underarm support (5) attached. The first support arm (3) includes the first belt connection plate (9), the first hinge section (11) and the first connection section (13).

The first belt connection plate (9) is a thin strip of material, located at a first end (15) of the first support arm (3), whose major axis is perpendicular to the centreline of the first support arm (3). The first belt connection plate (9) includes a plurality of belt slots (17), six in this case, which are thin essentially rectangular apertures through the first belt connection plate (9). The major axis of each belt slot (17) lies parallel to the centreline of the first support arm (3) in the rest position. In use the belt (2) is threaded through the belt slots (17) in such a way as to allow the position of the first support arm (3) on the belt (2) to be adjusted. This adjustment is most likely to be achieved by sliding the support arm (3,4) in question along the belt (2), the threading pattern through the belt slots (17) preventing movement once adjustment is completed.

The first belt connection plate (9) includes a first edge (18) and a second edge (19), each lying parallel to the major axis of the first belt connection plate (9), the first edge (18) being coincident with the first end (15) of the first support arm (3), and the second edge (19) being opposite the first edge (18).

The first hinge section (11) forms part of the second edge (19) of the first belt connection plate (9), the first hinge section (11) smoothly transitions into the first connection section (13). In use, the first hinge section (11) is configured to operate in a plane approximately parallel to the plane formed by the centreline of the first support arm (3) and the major axis of the first connection plate (9). The first hinge section (11) includes an elastic or spring loaded bias means (21) configured to maintain the centreline of the first support arm (3) in perpendicular relationship with the major axis of the first connection plate (9). The exposed end (22) of the first connection section (13) is a rod or bar. At a point near the distal end of the exposed end (22) an o-ring (23) or similar, retained in a groove (24) is present.

The spring loaded/elastic bias means (21) is of a known type (captured spring, flat elastic strap or similar) and allows a user to stretch upwards, disengaging from one or both underarm support (5,6) without the associated support arm (3,4) falling away. This means that no strap retaining the underarm support (5,6) in contact with the user is necessary. In other words the spring loaded/elastic bias means (21) is present to bias the support arm (3,4) to an upright position, maintaining the support arm essentially straight or in alignment.

Each underarm support (5,6) is trilobed, essentially equilateral in form, with a smooth symmetrical primary curved section (25), joining each adjacent lobe (30,31,32). Each primary curved section (25) includes a centrally located secondary curved section (26), which forms an indentation in the surface of the primary curved section (25). The radius of the primary curved section (25) is about 1.1 to 2 times the radius of the secondary curved section (26). The depth of the indentation formed by the secondary curved section (26) in the primary curved section (25) is between 5% and 15% of the secondary curved section's (26) radius. For example, if the radius of the secondary curved section (26) was 60 mm then the depth of the indentation formed by the secondary curved section (26) into the primary curved section (25), would be between 3 mm and 9 mm. The secondary curved section (26) has surprisingly been found to reduce the pressure applied by the back brace (1) to a user's underarm nerves and blood vessels in use. This has made the brace much more comfortable to wear for long periods. It has been found that a primary curved section (25) radius of 75 mm and a secondary curved section (26) radius of 60 mm works particularly well but other dimensions have also been found successful. Each of the underarm supports (5,6) is dished, such that the centre of the underarm support (5,6) lies on a different plane to that of the lobes (30,31,32).

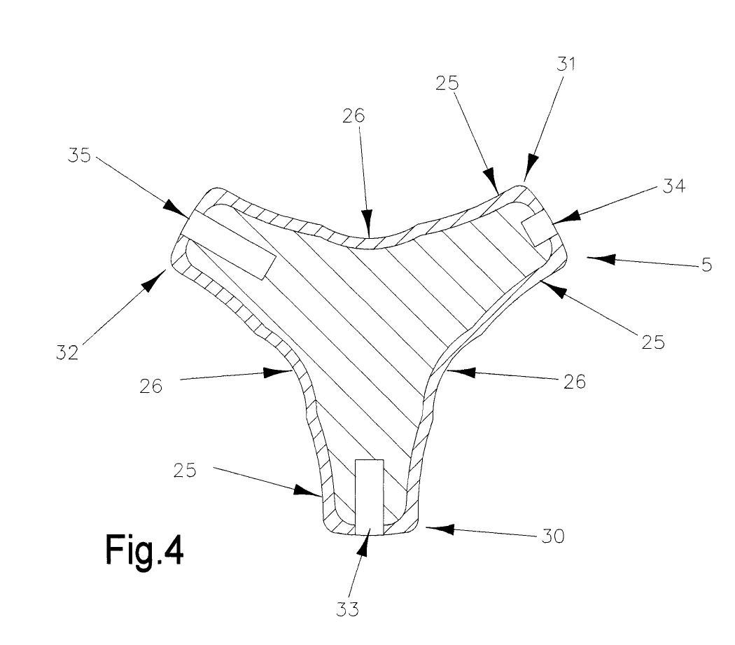

Referring to FIG. 4, a cross section through A-A of the first underarm support (5) is shown. Each lobe (30,31,32) includes an engagement socket (33,34,35), which is a hole extending along the centreline of the respective lobe (30,31,32). The depth of each engagement socket (33,34,35) is different, this allows a user to quickly adjust the overall length of the first support arm (3) between three preset lengths. It is envisioned that a user could manually modify one or more of the preset lengths by inserting a short piece of material into one or more of the engagement sockets (33,34,35). The underarm support is additionally padded for comfort.

In use, the belt (2) of the back brace (1) encircles a user's waist at approximately hip height, it is then tightened and the buckle (7) engaged. In this position each support arm (3,4) extends up to and under the respective arm of the user, the surface of the uppermost primary curved section (25) contacting the user's underarm. The dish in each underarm support (5,6) causing the uppermost lobes (30,31,32) to lean towards the users body. If the length of either support arm (3,4) is incorrect then the respective underarm support (5,6) can be disengaged from the relevant connection section (13,14) and an alternative engagement socket (33,34,35) used. When properly adjusted the back brace (1) extends the spine and supports it, allowing a user more movement and/or comfort.

In one embodiment the hinge sections (11,12) allow a user to bend forward but not backwards. In this case each hinge section (11,12) includes a stop means (not shown) of known type that prevents either connection section (13,14) from bending backwards. This may limit, but it does not prevent, a user from swivelling or twisting around. Each of the underarm supports (5,6) is free to swivel on its central axis, and each support arm (3,4) can bend forward; this allows the user to twist.

In use if a user stretches upwards to reach something the support arm (3,4) may disengage from under the user's arm, in this case the bias means (21) will bring the connection section (13,14) in question back to upright. The strength of the bias means (21) is such that it will return a support arm (3,4) to upright but will not prevent the normal movement of a user.

Though only a buckle (7) is mentioned as a means for releasably connecting the two ends of the belt (2) this is intended to encompass, hook and look connectors, magnetic catches, clips, pins, and any other means of achieving this.

The exposed end of each connection section (13,14) need not be circular in cross section, but may be e.g. hexagonal, Square, rectangular, octagonal or similar, and engage with a matching cross section engagement means (33,34,35). In this form each connection section (13,14) includes a swivel section (50,51) configured to allow the associated underarm support (5,6) to rotate axially. This section is required if the cross section of the exposed end (and/or associated engagement socket (33,34,35)) is not circular, for example hexagonal, octagonal, square, rectangular, oval or similar.

The different shaped engagement socket (33,34,35) could be used to allow customisation of the length of each of the support arms (3,4) of the back brace (1). A specific shaped extrusion could be produced and its profile protected, then this profile used for the exposed end (22) and engagement sockets (33,34,35).

Optionally each connection section (13,14) may include an adjustment section (52,53) which is a threaded section of the connection section (13,14). This adjustment section (52,53) allows the length of each support arm (3,4) to be adjusted.

In a preferred embodiment the hinge section (11,12) includes a stop means of known type which prevents the support arm (3,4) from bending past vertical in one direction. This stop means normally prevents the support arm (3,4) from bending towards the back of the user. Alternatively the hinge section (11,12) allows motion in two opposite directions. The stop means may be as simple as a physical feature that mechanically limits the range.

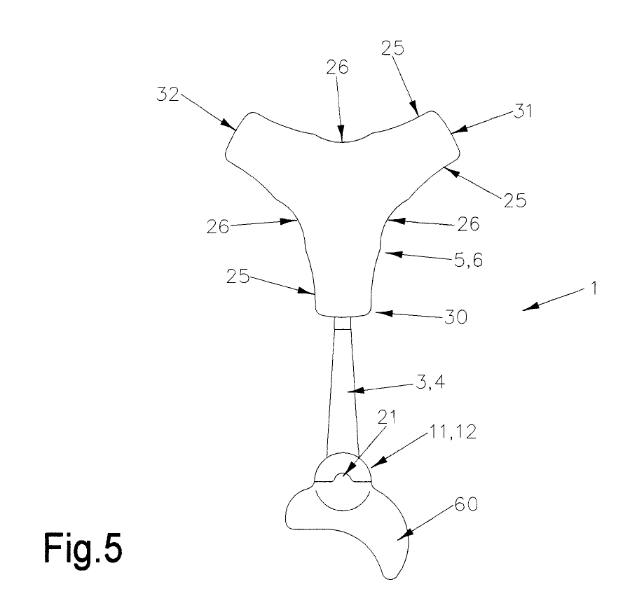

In a further embodiment, as shown in FIG. 5, the belt connection plates (9,10) are replaced with engagement sections (60) designed to engage with the top of the user's hips. The support arms (3,4) are prevented from disengaging from the user's hips by the shape of the engagement section (60) alone, or the engagement section (60) in combination with a close fitting garment.

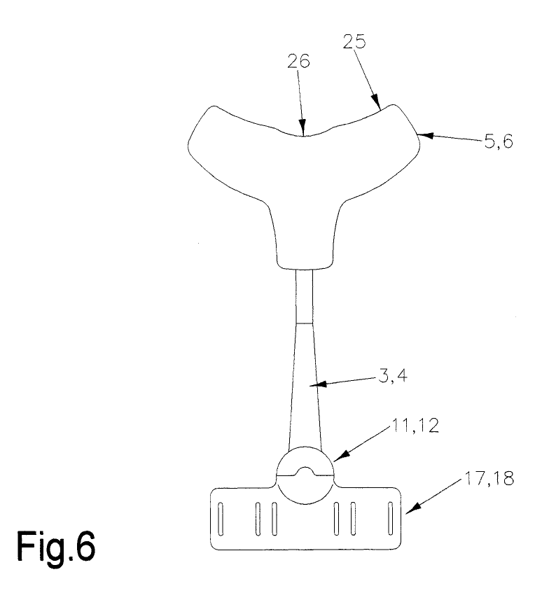

In a further embodiment as shown in FIG. 6 the underarm support (5,6) is not trilobed, it consist of a crutch that incorporates a single primary curved section (25) which includes a centrally located secondary curved section (26). The secondary curved section (26) forms an indentation in the surface of the primary curved section (25). The radius of the primary curved section (25) is about 1.1 to 2 times the radius of the secondary curved section (26). The depth of the indentation formed by the secondary curved section (26) in the primary curved section (25) is between 5% and 15% of the secondary curved section's (26) radius. For example, if the radius of the secondary curved section (26) was 60 mm then the depth of the indentation formed by the secondary curved section (26) into the primary curved section (25), would be between 3 mm and 9 mm. The secondary curved section (26) has surprisingly been found to reduce the pressure applied by the back brace (1) to a user's underarm nerves and blood vessels in use. This has made the brace much more comfortable to wear for long periods. It has been found that a primary curved section (25) radius of 75 mm and a secondary curved section (26) radius of 60 mm works particularly well but other dimensions have also been found successful.

In a further embodiment (not shown) one or more foam/resilient pads are releasably connected to the belt to further improve the comfort.

In further embodiments the underarm supports (5,6) are permanently attached, but still free to axially rotate on the support arms (3,4).

Any discussion of the prior art throughout the specification is not an admission that such prior art is widely known or forms part of the common general knowledge in the field.

1. A back brace including two support arms, each support arm including an underarm support located at one distal end, said underarm support is configured, when in use, to support an underarm of a user; wherein each support arm includes a hinge section, located close to or at the opposite distal end to that support arm's underarm support, each said hinge section incorporates a bias means configured to bias said support arm towards a predetermined alignment.

2. The back brace as claimed in claim 1 wherein the hinge section includes a stop means configured to prevents the associated support arm from pivoting in one direction.

3. The back brace as claimed in claim 2 wherein the stop means is configured to prevents, when the back brace is in use, the support arm from pivoting backwards.

4. The back brace as claimed in claim 1 wherein the bias means is configured to allow the associated support arm to move in two opposite directions and return it to a central position.

5. The back brace as claimed in claim 1 wherein an exposed distal end of each support arm closest to the hinge section includes a belt connection plate.

6. The back brace as claimed in claim 1 wherein the distal end of each support arm, said distal end being closest to the hinge section, includes an engagement section configured to releasably engage with a user's hip.

7. The back brace as claimed in claim 5 wherein the back brace includes a belt configured to releasably engage with the belt connection plate of each support arm.

8. The back brace as claimed in claim 1 wherein the underarm support is releasably attached to the respective support arm by a connection section.

9. The back brace as claimed in claim 8 wherein the connection section includes an exposed end that is configured and dimensioned to be inserted into a matching engagement socket.

10. The back brace as claimed in claim 1 wherein each underarm support includes at least one smooth primary curved section configured to engage with the underarm of the user when in use.

11. The back brace as claimed in claim 10 wherein each underarm support is trilobed, essentially equilateral in form, with one primary curved section joining each adjacent lobe.

12. The back brace as claimed in claim 11 wherein each underarm support is dished, such that the lobes are co-planar and the centre of the underarm support is distanced from that plane.

13. The back brace as claimed in claim 10 wherein the or each primary curved section is symmetrical.

14. The back brace as claimed in claim 10 wherein the or each primary curved section includes an essentially centrally located secondary curved section which forms an indentation in the surface of the primary curved section.

15. The back brace as claimed in claim 14 wherein the radius of the or each primary curved section is about 1.1 to 2 times the radius of the associated secondary curved section.

16. The back brace as claimed in claim 14 wherein the depth of the indentation formed by the secondary curved section in the or each primary curved section is between 5% and 15% of its radius.

17. The back brace as claimed in claim 16 wherein the depth of the indentation is 10%.

18. The back brace as claimed in claim 1 wherein each support arm includes a swivel section, said swivel section being configured to allow the axial rotation of the underarm support relative to the associated support arm.

19. The back brace as claimed in claim 11 wherein the or each primary curved section is symmetrical.

20. The back brace as claimed in claim 15 wherein the depth of the indentation formed by the secondary curved section in the or each primary curved section is between 5% and 15% of its radius.
